(19) United States
(12) Patent Application Publication
(10) Pub. No.: US 2024/0374701 A1
(43) Pub. Date: Nov. 14, 2024

(54) VACCINE COMPOSITIONS COMPRISING BRUCELLA STRAINS AND METHODS THEREOF

(71) Applicant: THE TEXAS A&M UNIVERSITY SYSTEM, College Station, TX (US)

(72) Inventors: Paul DE FIGEIREDO, College Station, TX (US); Jim SONG, College Station, TX (US); Tom FICHT, College Station, TX (US); Robert ALANIZ, College Station, TX (US)

(21) Appl. No.: 18/292,542

(22) PCT Filed: Jul. 28, 2022
(86) PCT No.: PCT/US2022/074252

§ 371 Date: Jan. 26, 2024

Related U.S. Application Data
(60) Provisional application No. 63/226,489, filed on Jul. 28, 2021.

(51) Int. Cl. A61K 39/02; A61K 39/00; A61K 45/06; A61P 35/00; A61P 37/04; C12N 1/20

(52) U.S. Cl. CPC A61K 39/098; A61K 45/06; A61P 35/00; A61P 37/04; C12N 1/205; A61K 2039/522; A61K 2039/572; A61K 2039/585

(57) ABSTRACT

The present disclosure provides pharmaceutical compositions comprising a live attenuated bacterial strain of Brucella melitensis, in particular a live attenuated bacterial strain of Brucella melitensis is Brucella melitensis 16M ΔvjbR (BmΔvjbR). Methods of utilizing the live attenuated bacterial strain of Brucella melitensis for treatment of a patient are also provided, including wherein the patient is in need of treatment for cancer, an autoimmune disorder, and/or an inflammatory disorder.

### CROSS-REFERENCE TO RELATED APPLICATIONS

This application claims the benefit under 35 USC § 119 (e) of U.S. Provisional Application Ser. No. 63/226,489, filed on Jul. 28, 2021, the entire disclosure of which is incorporated herein by reference.

### BACKGROUND AND SUMMARY OF THE INVENTION

The medical field is in a constant search for new therapeutic agents. Although recent advances in the treatment of cancer and diseases of inflammatory and autoimmune disorders are promising, further improvements are desperately needed. Recent research suggests that bacteria can surprisingly be effective agents for cancer and inflammatory/autoimmune treatments. However, these treatments are prone to severe side effects in patients that often lead to abandonment of the approach.

All previously used bacterial vectors have intrinsic deleterious or toxic features, and suboptimal safety profiles or routes of delivery that may significantly limit their broad utility in treatments. Observed negative features include an intraperitoneal route of delivery, viable microbe persistence in non-cancerous tissues when used for cancer treatment, significant endotoxin activity, pathogenic reversion potential, and limitations due to pre-existing host immunity.

Brucella melitensis is the etiological agent of brucellosis in livestock and wild animal populations. It is also the primary agent associated with human brucellosis, a disease marked by undulant fever and chronic symptoms. Over the past 30 years, live attenuated vaccine strains have been developed to protect animals against brucellosis. In particular, a novel class of attenuated mutants was created that comprise a deletion of the vjbR locus (BMEII1116) from Brucella melitensis 16M, referred to as BmΔvjbR. Importantly, this attenuated vaccine strain has displayed exceptional levels of safety following evaluation in tissue culture systems, as well as in immune-sufficient and immune-deficient mice, goats, sheep, and rhesus macaques.

The present disclosure provides a safe, live attenuated bacterial strain (BmΔvjbR) that can be utilized for treatment of various patient populations. In particular, the present disclosure describes use of the strain to provides advantageous effects in disease areas such as cancer, autoimmunity, and inflammation. As described herein, pharmaceutical compositions comprising the strain can be used as therapeutic tools to modulate immune response in various disease states and to provide advantageous strategies to improve or supplement existing immunotherapies.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows Heatmap showing the effect of live (Live) and heat-killed (HK) BmΔvjbR on the expression levels of immune related genes in mouse bone marrow developed macrophage (BMDM). Gene expression was compared using TaqMan Array, Mouse Immune (Applied Biosystems). The BMDM cells were incubated with either live or heat-killed (HK)BmΔvjbR for 1 hour and then rinsed three times in 1×PBS to remove extracellular bacteria prior to incubation in fresh Dulbecco's Modified Eagle's Medium (DMEM) medium. At 24 hours post inoculation, RNA was extracted from BMDM for gene expression assay. The bars indicate relative gene expression in the log 2 scale and the grade of up- and down-regulation are shown by purple and orange color gradation, respectively. The arrows highlight the genes, which were also analyzed using cytokine ELISA or flow cytometer. The quantitative log 2 fold change data is listed in the table.

FIG. 2A shows the heatmap showing the increased secretion of proinflammatory cytokines and chemokines from BmΔvjbR infected BMDMs at 24 hours post inoculation relative to PBS control. FIG. 2B shows the quantification of the selected cytokines and chemokines secreted by CD8⁺ T cells. FIG. 2C shows the live BmΔvjbR promotes expression of CD38 and 4-1BBL expression induced by live BmΔvjbR. FIG. 2D shows the quantification of CD38⁺ and 4-1BBL⁺ population among the infected or uninfected BMDMs. Data represent means±SEM (standard error of mean) from two independent experiments. *, **, ***: significance at p<0.05, 0.01, and 0.001, respectively.

FIG. 3A shows flow cytometric assessment of CD8⁺ T cells and representative dot-plots of CD69, TNFα and IFNγ. FIG. 3B shows the bar-graph representation of the dot-plots of CD69, TNFα, and IFNγ derived from three independent experiments. FIG. 3C shows the flowcytometric analysis and heatmap representation of activation, co-stimulation, and inflammatory cytokines of CD8⁺ T cells co-cultured with BmΔvjbR infected or non-infected macrophages for 3 days. The heatmap analysis was also conducted from flowcytometric dot-plots of restimulated CD8⁺ T cells. FIG. 3D shows the metabolic profile of CD8⁺ T cells was assessed by Seahorse metabolic assay measuring the glycolytic rate assay. The extracellular acidification rate is shown in the right panel followed by glycoPER in the left panel. **** p<0.0001 p<0.001, p<0.01, *, ** and ***: significance at p<0.05, 0.01 and 0.001, respectively. ns: not significant.

FIG. 4A shows the schematic diagram showing adoptive T cell therapy and BmΔvjbR treatment protocol. Mice were subcutaneously implanted with MC32-CEA cells followed by injection of BmΔvjbR on day 9 post injection of tumor cells. Subsequently, CAR-CEA transduced CD8⁺ T cells were adoptively transferred into the mice on day 12, and tumor size was measured every other day until the termination of the protocol at day 28. FIG. 4B shows vi-tsne plots of comparative immune cell populations in Ctrl and live BmΔvjbR treated tumor samples. FIG. 4C shows Neighborhood joining plots of Ctrl and live BmΔvjbR showing different immune cell populations of macrophages, dendritic cells, B cells and CD8⁺ T cells. The numbers 1-10 represent the cell clusters used for neighborhood analysis and heatmaps. Rows represent the cell phenotypes of interest whereas the columns represent the cell phenotypes in neighborhood in the heatmap. All the cell-to-cell interactions were assessed from the tumor sample with highly interacting neighbored cells shown in red whereas the avoided interactions shown in blue. FIG. 4D shows the reconstructed image of immune cells infiltration into tumor samples. FIG. 4E shows the quantification of macrophages, dendritic cells, and B cells in tumor samples. The markers representing the different immune cell populations are B220 (B cells), F4/80 (macrophages), CD11c (dendritic cells), Ki67 (proliferating cells) and CD8⁺ (CD8⁺ T cells).

FIG. 5A shows the survival of mice is significantly improved in the group of mice receiving BmΔvjbR from Day 18 onwards compared to control untreated group of mice. FIG. 5B shows the BmΔvjbR immunization followed by adoptive T-cell transfer significantly suppress the tumor growth from Day 15 post initiation of the experiment. FIG. 5C shows the H&E staining shows significant improvement in tumor in the group of mice receiving BmΔvjbR compared to control group. FIG. 5D shows the flow cytometry followed by graphical representation of infiltrating lymphocytes (Thy 1.2⁺ CD8⁺ T cells) confirm significantly higher infiltration of adoptively transferred CEA CD8⁺ T cells. FIG. 5E shows the confocal microscopy followed by graphical representation of infiltrating lymphocytes (Thy 1.2⁺ CD8⁺ T cells) confirm significantly higher infiltration of adoptively transferred CEA CD8⁺ T cells. FIG. 5F shows the representative immunofluorescence microscopy images show BmΔvjbR survival in tumor tissue after 19 days post injection. FIG. 5G shows the BmΔvjbR mainly colonizes in tumor. FIG. 5H shows BmΔvjbR can be observed in BMDMs with immunofluorescence microscopy after 1-, 4-, and 24-hour post inoculation (hpi). FIG. 5I shows the BmΔvjbR can be recovered from BMDMs, J774A.1, and RAW 264.7 macrophages at 1 hpi and 4 hpi, but no bacteria survived in these macrophages at 24 hpi. Data represent means±SEM from two independent experiments. ns: not significant; *, *, ***, ****: significance at p<0.05, 0.01, 0.001, and 0.0001, respectively.

FIG. 6 shows an exemplary plasmid construct suitable to produce the BmΔvjbR::tnaA strains, in which the tna gene was closed into a pBBR6Y-GFP vector and then transformed into BmΔvjbR to generate BmΔvjbR-tna.

FIGS. 7A-7I demonstrate that indole treatment dampens inflammation and promotes regulatory T cell (T_{reg}) expansion and activity. FIG. 7A: Representative flowcytometric dot-plot analysis of the effect of indole on CD11b⁺ cells. 0.25-, 0.5-, or 1.0-mM indole was dissolved in DMF for the representative experimental flowcytometric analysis. FIG. 7B: Graphical representation of flow cytometric dot-plots derived from three independent experiments of heat killed Salmonella Typhimurium (HKST) group. FIG. 7C: Flowcytometric histograms representing the indole dose-dependent differentiation of T_{regs}. Experiment (N=3) was performed under T_{reg} skew conditions ([TGF-β]=2 ng/ml, [IL-2]=100 U/mL). Th₀ control represents non-T_{reg} skew conditions. Butyrate was used as a control metabolite. FIG. 7D: Graphical representation of the effect of indole on the differentiation of T_{regs}. FIG. 7E: Graphical representation of the effects of indole alone on CIA in mice (N=5). FIG. 7F: Representative images of H&E, Safranin O (Saf-O) stained tissues, and confocal microscopy of knee tissues of CIA mice on day 60 post induction of arthritis. FIG. 7G: Quantitative analysis of H&E, Saf-O and T_{reg} infiltration from confocal microscopy sections of Control (Ctrl) and indole-treated mice. FIG. 7H: Flow cytometric dot-plot analysis of PD-1 and FoxP3 in ex vivo activated CD4⁺ T cells isolated from LNs and spleen of C57BL/6 mice. Exposure to indole drives these cells towards higher T_{reg} phenotype by increased FoxP3 expression. FIG. 7I: Graphical representation of FoxP3 derived from the flow cytometric dot-plots of CD4⁺ T cells exposed to indole. Graphical representation of PD-1⁺ FoxP3⁺ T cells (%) from the flow cytometric dot-plots. Data represent means±SD. Student's t-test or Tukey's multiple comparisons test was applied for statistical analysis. *, **, ***: significance at p<0.05, 0.01, 0.001.

FIGS. 8A-8E demonstrate that indole suppresses immune cell activation and BmΔvjBR is engineered to produce indole. FIG. 8A: Schematic representation of the engineered BmΔvjbR::tnaA harboring a plasmid carrying a tnaA expression cassette. The indole biosynthesis pathway is depicted in the figure. TnaA catalyzes the conversion of tryptophan to indole. FIG. 8B: Mass spectrometric analysis of indole production by engineered BmΔvjbR::tnaA. FIG. 8C: Western blotting analysis of the expression of tnaA protein in the parental strain compared with the engineered BmΔvjbR::tnaA strain. Graphical representation of the comparative analysis of indole production by BmΔvjBR parental bacterial strain and the engineered BmΔvjbR::tnaA strain. FIG. 8D: Colonization of engineered BmΔvjbR::tnaA in the spleen, liver, kidney and lymph-nodes of mice. The bacteria colonized in all the organs for 3 days post-inoculation and could be observed only in the spleen for 7 days. FIG. 8E: Serum ELISA analysis of anti-Brucella IgG production. The positive and negative controls were used as per the manufacturer's instructions.

FIGS. 9A-9G demonstrate that BmΔvjbR::tnaA significantly dampens inflammation and reduces arthritis in murine CIA model which is augmented by adoptive cell transfer (ACT) of T_{regs}. FIG. 9A: Cytokine arrays were used to measure pro-inflammatory cytokines produced by control, BmΔvjbR, and BmΔvjbR::tnaA treated BMDMs. FIG. 9B: Flow cytometric analysis of IFN-γ and TNF-α of T cells co-cultured with BMDMs. BMDMs were treated with either BmΔvjbR::tnaA or BmΔvjbR and then co-cultured with CD4⁺ T cells derived from pooled LNs and spleen of C57BL/6 mice for the assay. FIG. 9C: Arthritis score and arthritis incidence in CIA C57BL/6 mice from control (Ctrl); BmΔvjbR; BmΔvjbR::tnaA; BmΔvjbR::tnaA followed by ACT of T_{regs} (T_{reg}) and ACT of T_{regs} only; (N=5 in each group). FIG. 9D: Representative images of H&E, Saf-O staining, and confocal microscopy from mouse knees on day 60 post CIA induction. Quantitative analysis of T_{reg} infiltration and inflammation scores from these mice are also shown. FIG. 9E: Cells from the LNs and spleen were collected from CIA-induced mouse groups (Ctrl, BmΔvjbR::tnaA, and BmΔvjbR::tnaA combined with ACT of T_{regs}. These cells were then stained and quantified by flow cytometry using markers for CD4⁺ T cells and intracellular staining of FoxP3 (T_{regs}). FIG. 9F: CIA-induced mice were treated with PBS (Ctrl), ACT of T_{regs} only (T_{regs} only; N=5), or BmΔvjbR::tnaA combined with ACT of T_{regs} (N=5). Cells from the knee and ankle joints were stained with 21 markers and measured by CyTEK aurora flow cytometry. Heatmap shows immune cell profiles in different treatment groups of mice (scale bar represents percentage of cell in each treatment group within each cell type). FIG. 9G: viSNE map shows the four subtypes of B cells differentially expressed in the treated group of mice. Data represent means±SD. Student's t-test or Tukey's multiple comparisons test was applied for statistical analysis. *, **, ***: significance at p<0.05, 0.01, 0.001.

### DETAILED DESCRIPTION

Various embodiments of the invention are described herein as follows. In an illustrative aspect, a pharmaceutical composition comprising a live attenuated bacterial strain of Brucella melitensis is provided. In another illustrative aspect, a method of treating a patient is provided. The method comprises the step of administering a pharmaceutical composition comprising a live attenuated bacterial strain of Brucella melitensis to the patient.

In an embodiment, the live attenuated bacterial strain of Brucella melitensis is Brucella melitensis 16M ΔvjbR (BmΔvjbR). Brucella melitensis 16M is available, for instance, as ATCC #23456. Methods of making and obtaining BmΔvjbR have been previously described, for instance in Arenas-Gamboa et al., Clin Vaccine Immunol, 2012; 19:249-60, de Figueiredo et al., Am J Pathol, 2015; 185:1505-17, Pandey et al., Methods Mol Biol, 2014; 1197:229-44, and Pandey et al., Front Cell Infect Microbiol, 2018; 8:103, each of which are incorporated herein in their entirety.

In an embodiment, the pharmaceutical composition is an oral formulation. In an embodiment, the oral formulation is selected from the group consisting of a tablet, a capsule, a suspension, an emulsion, a syrup, a colloidal dispersion, a dispersion, and an effervescent composition. In an embodiment, the oral formulation is a tablet. In an embodiment, the oral formulation is a capsule. In an embodiment, the oral formulation is a suspension. In an embodiment, the oral formulation is an emulsion. In an embodiment, the oral formulation is a syrup. In an embodiment, the oral formulation is a colloidal dispersion. In an embodiment, the oral formulation is a dispersion. In an embodiment, the oral formulation is an effervescent composition.

In an embodiment, the pharmaceutical composition is a parenteral formulation. In an embodiment, the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous. In an embodiment, the parenteral formulation is intravenous. In an embodiment, the parenteral formulation is intraarterial. In an embodiment, the parenteral formulation is intraperitoneal. In an embodiment, the parenteral formulation is intrathecal. In an embodiment, the parenteral formulation is intradermal. In an embodiment, the parenteral formulation is epidural. In an embodiment, the parenteral formulation is intracerebroventricular. In an embodiment, the parenteral formulation is intraurethral. In an embodiment, the parenteral formulation is intrasternal. In an embodiment, the parenteral formulation is intracranial. In an embodiment, the parenteral formulation is intratumoral. In an embodiment, the parenteral formulation is intramuscular. In an embodiment, the parenteral formulation is subcutaneous.

In an embodiment, the pharmaceutical composition comprises one or more pharmaceutically acceptable carriers.

In an embodiment, the pharmaceutical composition is formulated as a single dose. In an embodiment, the pharmaceutical composition is formulated as a single unit dose. As used herein, the term “unit dose” is a discrete amount of the pharmaceutical composition comprising a predetermined amount bacterial strain. According to the present disclosure, the terms “single dose” and “single unit dose” include embodiments wherein the pharmaceutical composition can be administered as a single parenteral injection or administered as multiple parenteral injections. In one embodiment, a single dose or single unit dose of the pharmaceutical composition can be parenterally administered to a patient at one location on the patient's body. In another embodiment, a single dose or single unit dose of the pharmaceutical composition can be parenterally administered to an animal in multiple injections at a single location on the patient's body. In yet another embodiment, a single dose or single unit dose of the pharmaceutical composition can be parenterally administered to a patient in multiple injections at more than one location on the patient's body. In embodiments wherein multiple injections of the pharmaceutical composition are utilized, the multiple injections can be administered to the animal over a reasonable duration of time.

In an embodiment, the pharmaceutical composition further comprises a second therapeutic agent. In an embodiment, the second therapeutic agent is an anti-cancer therapy. In an embodiment, the second therapeutic agent is an auto-immune therapy. In an embodiment, the second therapeutic agent is an anti-inflammatory therapy.

In an embodiment, the pharmaceutical composition further comprises indole. In an embodiment, the live attenuated bacterial strain of Brucella melitensis is modified to produce indole. In an embodiment, an indole producing attenuated strain of Brucella melitensis is provided. In an embodiment, the Brucella melitensis strain harbors a mutation in one of its virulence genes, such as vjbR, asp14, and mucR that inactivates the virulence gene, and includes an exogenously introduce gene (e.g., tnaA) that encodes for tryptophanase activity. As used herein, the term tryptophanase encompasses any protein that catalyzes the hydrolysis of tryptophan to produce indole, pyruvate, and ammonium. In an embodiment, the mutation of the virulence gene is a deletion mutation. In an embodiment, the attenuated strain of Brucella melitensis harbors a deletion in the vjbR gene and the gene expressing the enzyme tryptophanase (tnaA) is under the control of a constitutive promoter. In an embodiment, the attenuated strain of Brucella melitensis is BmrvjbR. In an embodiment, the attenuated strain of Brucella melitensis is transfected with a gene encoding tryptophanase, optionally wherein the tryptophanase encoding gene is the E. coli tnaA gene. In an embodiment, the attenuated strain of Brucella melitensis is BmΔvjbR that has been transfected with a plasmid that comprises the E. coli tnaA gene under the control of a constitutive promoter, optionally wherein the promoter is J23119 (SpeI). The plasmid can include additional selectable marker and reporter genes, including for example a Green Fluorescent Protein encoding gene. One plasmid construct suitable to produce the BmΔvjbR::tnaA strains of the present invention is provided in FIG. 6.

In accordance with one embodiment, a method is provided for using of live attenuated Brucella melitensis as an immuno-modulator, wherein the metabolism of the attenuated Brucella melitensis has been further reprogrammed to amplify antiautoimmune/inflammation activity. In accordance with one embodiment, an indole producing attenuated BmΔvjbR strain is provided for administration to subjects in need of immunomodulation. In one embodiment, the method comprises the administration to a subject in need of immunomodulation a composition comprising an attenuated strain of Brucella melitensis that harbors a mutation in one of its virulence genes, such as vjbR, asp14, and mucR and constitutively expresses an exogenously introduced tryptophanase (tnaA) gene. In one embodiment, the attenuated strain of Brucella melitensis harbors a deletion in vjbR and expresses tryptophanase (tnaA) under the control of a constitutive promoter

In an illustrative embodiment, a method of treating a patient is provided. The method comprises the step of administering a pharmaceutical composition comprising a live attenuated bacterial strain of Brucella melitensis to the patient. In an embodiment, the live attenuated bacterial strain of Brucella melitensis is Brucella melitensis 16M ΔvjbR (BmΔvjbR). Any of the embodiments of the pharmaceutical as described previously can be utilized in the methods of treating a patient.

In an embodiment, the patient is in need of treatment for cancer. In an embodiment, the cancer is selected from the group consisting of melanoma, breast cancer, prostate cancer, pancreatic cancer, and colorectal cancer. In an embodiment, the cancer is melanoma. In an embodiment, the cancer is breast cancer. In an embodiment, the cancer is prostate cancer. In an embodiment, the cancer is pancreatic cancer. In an embodiment, the cancer is colorectal cancer. In an embodiment, the cancer is resistant to a chimeric antigen receptor (CAR)-T cell therapy.

In an embodiment, the patient is in need of treatment for an autoimmune disorder. In an embodiment, the autoimmune disorder is colitis. In an embodiment, the autoimmune disorder is inflammatory bowel disease.

In an embodiment, the patient is in need of treatment for an inflammatory disorder. In an embodiment, the inflammatory disorder is colitis. In an embodiment, the inflammatory disorder is inflammatory bowel disease.

In an embodiment, the pharmaceutical composition is administered to the patient at a dose of about 0.001 to about 1000 mg of active ingredient per kg of patient body weight. The “active ingredient” in this context refers to the live attenuated bacterial strain of Brucella melitensis. In an embodiment, the pharmaceutical composition is administered to the patient at a dose of about 0.001 to about 100 mg of active ingredient per kg of patient body weight. In an embodiment, the pharmaceutical composition is administered to the patient at a dose of dose of about 0.01 to about 100 mg of active ingredient per kg of patient body weight. In an embodiment, the pharmaceutical composition is administered to the patient at a dose of about 0.1 to about 100 mg of active ingredient per kg of patient body weight. In an embodiment, the pharmaceutical composition is administered to the patient at a dose of about 0.1 to about 10 mg of active ingredient per kg of patient body weight. In an embodiment, the pharmaceutical composition is administered to the patient at a dose of a dose of about 1 to about 5 mg of active ingredient per kg of patient body weight.

In an embodiment, the method elicits a CD8⁺ T cell response in the patient. In an embodiment, the method elicits a CD4⁺ T cell response in the patient. In an embodiment, the method elicits a T regulatory cell response in the patient. In an embodiment, the method increases PD-1 expression on CD8⁺ T cells in the patient.

In an embodiment, the method increases the number of CAR-T cells in a tumor microenvironment of the patient. In an embodiment, the method increases the activity of CAR-T cells in a tumor microenvironment of the patient. In an embodiment, the method modifies a tumor microenvironment of the patient to a pro-inflammatory state.

In an embodiment, the method modifies a tumor microenvironment of the patient by increasing macrophages in the tumor microenvironment. In an embodiment, the macrophages are proliferating macrophages. In an embodiment, the macrophages are non-proliferating macrophages. In an embodiment, the method modifies a tumor microenvironment of the patient by increasing dendritic cells in the tumor microenvironment.

In an embodiment, the method modifies a tumor microenvironment of the patient by increasing CD8⁺ PD-1-T cells in the tumor microenvironment. In an embodiment, the method promotes pro-inflammatory M1 polarization of macrophages in the patient.

In an embodiment, the method induces macrophages in the patient to express a pro-inflammatory cytokine/chemokine. In an embodiment, the pro-inflammatory cytokine/chemokine is selected from the group consisting of IL-6, IL-1α, IL-12b (IL12p40), Cc15 (RANTES), Cxc110 (IP-10), Ccl2 (MCP-1), and Ccl3 (MIP-1α). In an embodiment, the pro-inflammatory cytokine/chemokine is IL-6. In an embodiment, the pro-inflammatory cytokine/chemokine is IL-1α. In an embodiment, the pro-inflammatory cytokine/chemokine is IL-12b (IL12p40). In an embodiment, the pro-inflammatory cytokine/chemokine is Cc15 (RANTES). In an embodiment, the pro-inflammatory cytokine/chemokine is Cxc110 (IP-10). In an embodiment, the pro-inflammatory cytokine/chemokine is Cc12 (MCP-1). In an embodiment, the pro-inflammatory cytokine/chemokine is Cc13 (MIP-1α). In an embodiment, the method induces reduction of VEGF in the patient.

In an embodiment, the method enhances inflammatory potential of CD-8⁺ T cells. In an embodiment, the enhanced inflammatory potential is an increased production of TNFα from CD8⁺ T cells. In an embodiment, the enhanced inflammatory potential is an increased production of IFNγ from CD8⁺ T cells. In an embodiment, the enhanced inflammatory potential is an increased production of IL-2 from CD8⁺ T cells. In an embodiment, the enhanced inflammatory potential is an increased expression of OX40 in CD8⁺ T cells. In an embodiment, the enhanced inflammatory potential is an increased expression of 4-1BB in CD8⁺ T cells.

In an illustrative aspect, a transgenic attenuated Brucella melitensis strain is provide. The strain comprises a mutation in a virulence gene of said strain, said mutation selected from the group consisting of vjbR, asp14, and mucR wherein said mutation inactivates the virulence gene; and a nucleic acid encoding tryptophanase (tnaA). In an embodiment, the mutated virulence gene is vjbR, and the nucleic acid encoding tnuA is expressed under the control of a constitutive promoter. In an embodiment, the mutated virulence gene is BmΔvjbR and the nucleic acid encoding tnaA comprises E. coli tnaA.

The following numbered embodiments are contemplated and are non-limiting:

1. A pharmaceutical composition comprising a live attenuated bacterial strain of Brucella melitensis.
2. The pharmaceutical composition of clause 1, any other suitable clause, or any combination of suitable clauses, wherein the live attenuated bacterial strain of Brucella melitensis is Brucella melitensis 16M ΔvjbR (BmΔvjbR).
3. The pharmaceutical composition of clause 1, any other suitable clause, or any combination of suitable clauses, wherein the pharmaceutical composition is an oral formulation.
4. The pharmaceutical composition of clause 3, any other suitable clause, or any combination of suitable clauses, wherein the oral formulation is selected from the group consisting of a tablet, a capsule, a suspension, an emulsion, a syrup, a colloidal dispersion, a dispersion, and an effervescent composition.
5. The pharmaceutical composition of clause 3, any other suitable clause, or any combination of suitable clauses, wherein the oral formulation is a tablet.
6. The pharmaceutical composition of clause 3, any other suitable clause, or any combination of suitable clauses, wherein the oral formulation is a capsule.
7. The pharmaceutical composition of clause 3, any other suitable clause, or any combination of suitable clauses, wherein the oral formulation is a suspension.
8. The pharmaceutical composition of clause 3, any other suitable clause, or any combination of suitable clauses, wherein the oral formulation is an emulsion.
9. The pharmaceutical composition of clause 3, any other suitable clause, or any combination of suitable clauses, wherein the oral formulation is a syrup.
10. The pharmaceutical composition of clause 3, any other suitable clause, or any combination of suitable clauses, wherein the oral formulation is a colloidal dispersion.
11. The pharmaceutical composition of clause 3, any other suitable clause, or any combination of suitable clauses, wherein the oral formulation is a dispersion.
12. The pharmaceutical composition of clause 3, any other suitable clause, or any combination of suitable clauses, wherein the oral formulation is an effervescent composition.
13. The pharmaceutical composition of clause 1, any other suitable clause, or any combination of suitable clauses, wherein the pharmaceutical composition is a parenteral formulation.
14. The pharmaceutical composition of clause 13, any other suitable clause, or any combination of suitable clauses, wherein the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
15. The pharmaceutical composition of clause 13, any other suitable clause, or any combination of suitable clauses, wherein the parenteral formulation is intravenous.
16. The pharmaceutical composition of clause 13, any other suitable clause, or any combination of suitable clauses, wherein the parenteral formulation is intraarterial.
17. The pharmaceutical composition of clause 13, any other suitable clause, or any combination of suitable clauses, wherein the parenteral formulation is intraperitoneal.
18. The pharmaceutical composition of clause 13, any other suitable clause, or any combination of suitable clauses, wherein the parenteral formulation is intrathecal.
19. The pharmaceutical composition of clause 13, any other suitable clause, or any combination of suitable clauses, wherein the parenteral formulation is intradermal.
20. The pharmaceutical composition of clause 13, any other suitable clause, or any combination of suitable clauses, wherein the parenteral formulation is epidural.
21. The pharmaceutical composition of clause 13, any other suitable clause, or any combination of suitable clauses, wherein the parenteral formulation is intracerebroventricular.
22. The pharmaceutical composition of clause 13, any other suitable clause, or any combination of suitable clauses, wherein the parenteral formulation is intraurethral.
23. The pharmaceutical composition of clause 13, any other suitable clause, or any combination of suitable clauses, wherein the parenteral formulation is intrasternal.
24. The pharmaceutical composition of clause 13, any other suitable clause, or any combination of suitable clauses, wherein the parenteral formulation is intracranial.
25. The pharmaceutical composition of clause 13, any other suitable clause, or any combination of suitable clauses, wherein the parenteral formulation is intratumoral.
26. The pharmaceutical composition of clause 13, any other suitable clause, or any combination of suitable clauses, wherein the parenteral formulation is intramuscular.
27. The pharmaceutical composition of clause 13, any other suitable clause, or any combination of suitable clauses, wherein the parenteral formulation is subcutaneous.
28. The pharmaceutical composition of clause 1, any other suitable clause, or any combination of suitable clauses, wherein the pharmaceutical composition comprises one or more pharmaceutically acceptable carriers.
29. The pharmaceutical composition of clause 1, any other suitable clause, or any combination of suitable clauses, wherein the pharmaceutical composition is formulated as a single dose.
30. The pharmaceutical composition of clause 1, any other suitable clause, or any combination of suitable clauses, wherein the pharmaceutical composition is formulated as a single unit dose.
31. The pharmaceutical composition of clause 1, any other suitable clause, or any combination of suitable clauses, wherein the pharmaceutical composition further comprises a second therapeutic agent.
32. The pharmaceutical composition of clause 31, any other suitable clause, or any combination of suitable clauses, wherein the second therapeutic agent is an anti-cancer therapy.
33. The pharmaceutical composition of clause 31, any other suitable clause, or any combination of suitable clauses, wherein the second therapeutic agent is an auto-immune therapy.
34. The pharmaceutical composition of clause 31, any other suitable clause, or any combination of suitable clauses, wherein the second therapeutic agent is an anti-inflammatory therapy.
35. The pharmaceutical composition of clause 1, any other suitable clause, or any combination of suitable clauses, wherein the pharmaceutical composition further comprises indole.
36. The pharmaceutical composition of clause 35, any other suitable clause, or any combination of suitable clauses, wherein the live attenuated bacterial strain of Brucella melitensis is modified to produce indole.
37. A method of treating a patient, said method comprising the step of administering a pharmaceutical composition comprising a live attenuated bacterial strain of Brucella melitensis to the patient.
38. The method of clause 37, any other suitable clause, or any combination of suitable clauses, wherein the live attenuated bacterial strain of Brucella melitensis is Brucella melitensis 16M ΔvjbR (BmΔvjbR).
39. The method of clause 37, any other suitable clause, or any combination of suitable clauses, wherein the patient is in need of treatment for cancer.
40. The method of clause 39, any other suitable clause, or any combination of suitable clauses, wherein the cancer is selected from the group consisting of melanoma, breast cancer, prostate cancer, pancreatic cancer, and colorectal cancer.
41. The method of clause 39, any other suitable clause, or any combination of suitable clauses, wherein the cancer is melanoma.
42. The method of clause 39, any other suitable clause, or any combination of suitable clauses, wherein the cancer is breast cancer.
43. The method of clause 39, any other suitable clause, or any combination of suitable clauses, wherein the cancer is prostate cancer.
44. The method of clause 39, any other suitable clause, or any combination of suitable clauses, wherein the cancer is pancreatic cancer.
45. The method of clause 39, any other suitable clause, or any combination of suitable clauses, wherein the cancer is colorectal cancer.
46. The method of clause 39, any other suitable clause, or any combination of suitable clauses, wherein the cancer is resistant to a chimeric antigen receptor (CAR)-T cell therapy.
47. The method of clause 37, any other suitable clause, or any combination of suitable clauses, wherein the patient is in need of treatment for an autoimmune disorder.
48. The method of clause 47, any other suitable clause, or any combination of suitable clauses, wherein the autoimmune disorder is colitis.
49. The method of clause 47, any other suitable clause, or any combination of suitable clauses, wherein the autoimmune disorder is inflammatory bowel disease.
50. The method of clause 37, any other suitable clause, or any combination of suitable clauses, wherein the patient is in need of treatment for an inflammatory disorder.
51. The method of clause 50, any other suitable clause, or any combination of suitable clauses, wherein the inflammatory disorder is colitis.
52. The method of clause 50, any other suitable clause, or any combination of suitable clauses, wherein the inflammatory disorder is inflammatory bowel disease.
53. The method of clause 37, any other suitable clause, or any combination of suitable clauses, wherein the pharmaceutical composition is administered to the patient at a dose of about 0.001 to about 1000 mg of active ingredient per kg of patient body weight.
54. The method of clause 37, any other suitable clause, or any combination of suitable clauses, wherein the pharmaceutical composition is administered to the patient at a dose of about 0.001 to about 100 mg of active ingredient per kg of patient body weight.
55. The method of clause 37, any other suitable clause, or any combination of suitable clauses, wherein the pharmaceutical composition is administered to the patient at a dose of dose of about 0.01 to about 100 mg of active ingredient per kg of patient body weight.
56. The method of clause 37, any other suitable clause, or any combination of suitable clauses, wherein the pharmaceutical composition is administered to the patient at a dose of about 0.1 to about 100 mg of active ingredient per kg of patient body weight.
57. The method of clause 37, any other suitable clause, or any combination of suitable clauses, wherein the pharmaceutical composition is administered to the patient at a dose of about 0.1 to about 10 mg of active ingredient per kg of patient body weight.
58. The method of clause 37, any other suitable clause, or any combination of suitable clauses, wherein the pharmaceutical composition is administered to the patient at a dose of a dose of about 1 to about 5 mg of active ingredient per kg of patient body weight.
59. The method of clause 37, any other suitable clause, or any combination of suitable clauses, wherein the method elicits a CD8+ T cell response in the patient.
60. The method of clause 37, any other suitable clause, or any combination of suitable clauses, wherein the method elicits a CD4+ T cell response in the patient.
61. The method of clause 37, any other suitable clause, or any combination of suitable clauses, wherein the method elicits a T regulatory cell response in the patient.
62. The method of clause 37, any other suitable clause, or any combination of suitable clauses, wherein the method increases PD-1 expression on CD8+ T cells in the patient.
63. The method of clause 37, any other suitable clause, or any combination of suitable clauses, wherein the method increases the number of CAR-T cells in a tumor microenvironment of the patient.
64. The method of clause 37, any other suitable clause, or any combination of suitable clauses, wherein the method increases the activity of CAR-T cells in a tumor microenvironment of the patient.
65. The method of clause 37, any other suitable clause, or any combination of suitable clauses, wherein the method modifies a tumor microenvironment of the patient to a pro-inflammatory state.
66. The method of clause 37, any other suitable clause, or any combination of suitable clauses, wherein the method modifies a tumor microenvironment of the patient by increasing macrophages in the tumor microenvironment.
67. The method of clause 66, any other suitable clause, or any combination of suitable clauses, wherein the macrophages are proliferating macrophages.
68. The method of clause 66, any other suitable clause, or any combination of suitable clauses, wherein the macrophages are non-proliferating macrophages.
69. The method of clause 37, any other suitable clause, or any combination of suitable clauses, wherein the method modifies a tumor microenvironment of the patient by increasing dendritic cells in the tumor microenvironment.
70. The method of clause 37, any other suitable clause, or any combination of suitable clauses, wherein the method modifies a tumor microenvironment of the patient by increasing CD8+PD-1-T cells in the tumor microenvironment.
71. The method of clause 37, any other suitable clause, or any combination of suitable clauses, wherein the method promotes pro-inflammatory M1 polarization of macrophages in the patient.
72. The method of clause 37, any other suitable clause, or any combination of suitable clauses, wherein the method induces macrophages in the patient to express a pro-inflammatory cytokine/chemokine.
73. The method of clause 72, any other suitable clause, or any combination of suitable clauses, wherein the pro-inflammatory cytokine/chemokine is selected from the group consisting of IL-6, IL-1α, IL-12b (IL12p40), Cc15 (RANTES), Cxc110 (IP-10), Ccl2 (MCP-1), and Ccl3 (MIP-1α).
74. The method of clause 72, any other suitable clause, or any combination of suitable clauses, wherein the pro-inflammatory cytokine/chemokine is IL-6.
75. The method of clause 72, any other suitable clause, or any combination of suitable clauses, wherein the pro-inflammatory cytokine/chemokine is IL-1α.
76. The method of clause 72, any other suitable clause, or any combination of suitable clauses, wherein the pro-inflammatory cytokine/chemokine is IL-12b (IL12p40).
77. The method of clause 72, any other suitable clause, or any combination of suitable clauses, wherein the pro-inflammatory cytokine/chemokine is Cc15 (RANTES).
78. The method of clause 72, any other suitable clause, or any combination of suitable clauses, wherein the pro-inflammatory cytokine/chemokine is Cxc110 (IP-10).
79. The method of clause 72, any other suitable clause, or any combination of suitable clauses, wherein the pro-inflammatory cytokine/chemokine is Ccl2 (MCP-1).
80. The method of clause 72, any other suitable clause, or any combination of suitable clauses, wherein the pro-inflammatory cytokine/chemokine is Cc13 (MIP-1α).
81. The method of clause 37, any other suitable clause, or any combination of suitable clauses, wherein the method induces reduction of VEGF in the patient.
82. The method of clause 37, any other suitable clause, or any combination of suitable clauses, wherein the method enhances inflammatory potential of CD-8+ T cells.
83. The method of clause 82, any other suitable clause, or any combination of suitable clauses, wherein the enhanced inflammatory potential is an increased production of TNFα from CD8⁺ T cells.
84. The method of clause 82, any other suitable clause, or any combination of suitable clauses, wherein the enhanced inflammatory potential is an increased production of IFNγ from CD8⁺ T cells.
85. The method of clause 82, any other suitable clause, or any combination of suitable clauses, wherein the enhanced inflammatory potential is an increased production of IL-2 from CD8⁺ T cells.
86. The method of clause 82, any other suitable clause, or any combination of suitable clauses, wherein the enhanced inflammatory potential is an increased expression of OX40 in CD8⁺ T cells.
87. The method of clause 82, any other suitable clause, or any combination of suitable clauses, wherein the enhanced inflammatory potential is an increased expression of 4-1BB in CD8⁺ T cells.
88. A transgenic attenuated Brucella melitensis strain, said strain comprising a mutation in a virulence gene of said strain, said mutation selected from the group consisting of vjbR, asp14, and mucR wherein said mutation inactivates the virulence gene; and a nucleic acid encoding tryptophanase (tnaA).
89. The transgenic attenuated Brucella melitensis strain of clause 88, any other suitable clause, or any combination of suitable clauses, wherein the mutated virulence gene is vjbR, and the nucleic acid encoding tnaA is expressed under the control of a constitutive promoter.
90. The transgenic attenuated Brucella melitensis strain of clause 88, any other suitable clause, or any combination of suitable clauses, wherein the mutated virulence gene is BmΔvjbR and the nucleic acid encoding tnaA comprises E. coli tnaA.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Exemplary Experimental Procedures

The instant example provides exemplary materials and methods utilized in Examples 2-6 as described herein.

Bacterial culture and inoculation. To prepare BmΔvjbR for in vitro inoculation and in vivo injection, BmΔvjbR was streaked on a tryptone soya agar (TSA) plate and incubated at 37° C. for 3 days until single-isolated colonies were obtained. Prior to the experiment, single colony of BmΔvjbR from the TSA plate was inoculated into 2 ml of tryptone soya broth (TSB) culture tube and incubated at 37° C. with shaking (250 rpm) for 24 hours. Before inoculation in mice, the bacteria were centrifuged at (10,000× g, 1 minute) and washed twice with 1× phosphate-buffered saline (PBS, pH 7.4, unless otherwise indicated). The bacterial pellet was resuspended into 1×PBS up to OD600 of 1.0 (˜ 5×10⁹ CFUs/ml). For in vitro inoculation, bacteria were added in each well of a 24 well plate with macrophage monolayer at MOI (multiplicity of infection) of 20 in Dulbecco's Modified Eagle's Medium (DMEM) (Thermo Fisher Scientific), and the plate was centrifuged at 500×g for 5 minutes to enhance bacterial interaction with the macrophages. Following inoculation, the macrophages were incubated at 37° C. for 30 minutes to allow the uptake of bacteria. After 30 minutes of incubation, the non-internalized bacteria were removed by washing the cell monolayer twice with warm PBS and then fresh DMEM medium containing 50 μg/mL of gentamicin was added into each well for continue cultivation until assay. For in vivo animal experiment, at 9 days post inoculation of tumor cells in mice, 5×10⁷ CFUs of BmΔvjbR in 100 μl of 1×PBS was intravenously injected into each mouse.

Macrophage cultures. For generation of murine BMDMs, bone marrow cells were harvested from the tibia and femur of 6-8 weeks C57BL/6 mice under sterile conditions. Red blood cells were removed from bone marrow by using sterile Red Blood Cell Lysing Buffer (0.8% NH₄Cl). The bone marrow cells (1×10⁷ cells/plate) were then seeded onto a 15 cm petri dish in DMEM containing 10% FBS, 10 ng/ml mouse M-CSF (PeproTech, Inc) and supplemented with penicillin-streptomycin (100 IU/ml and 100 μg/ml) (Sigma). At day 3, non-adherent cells were removed by replacing with fresh medium, and adherent macrophages were cultured in the fresh medium for another 4 days, replacing half of the medium with fresh medium on days 5 and 6, before use. For BMDM expansion, cells can be retrieved in cold 1×PBS by vigorously pipet on day 5 and then re-seeded in extra culture plates for growing an additional 2 days before use. Murine RAW264.7 (ATCC TIB-71) and J774A.1 (ATCC TIB-67) macrophage cell lines were both cultured according to ATCC's recommendations in DMEM media containing 10% FBS and penicillin-streptomycin (100 IU/ml and 100 μg/ml).

Cytokine responses. BMDMs were seeded in the wells of 24-well plates at a concentration of 2.0×10⁵ cells/well in 1.0 mL of DMEM without antibiotics. After overnight culture, the cells were inoculated with heat killed or live BmΔvjbR bacteria at a MOI of 20. At 24 hours post-treatment, cellular supernatant was collected and removed cell debris by centrifugation, and analyzed for the presence of cytokines/chemokines like GM-CSF, IFNγ, IL-1β, IL-2, IL-3, IL-5, IL-6, IL-9, IL-17, IP-10/CCL10, KC/CXCLI1, MCP-1/CCL2, M-CSF, MIP-1α/CCL3, MIP-1β/CCL4, MIP-2/CXCL2, RANTES/CCL5, TNFα, and VEGF by using a Multiplex Mouse Cytokine/Chemokine Array 31-Plex technology (MD31, Eve Technologies).

Flow cytometric analysis. CD8⁺ T cells, isolated by using mouse CD8⁺ T cell isolating kit (BioLegend), were co-cultured in vitro with BmΔvjbR treated macrophages. The CD8⁺ T cells were then analyzed by flow cytometry following specific gating CD8⁺ marker and exclusion of dead cells by using Aqua Zombie NIR staining dye (BioLegend). The CD8⁺ T cells markers of PD-1, CD69, 4-1BB, CD27, CD62L and OX40 were assessed either immediately post-co-culture with infected BMDMs or 3 days after re-stimulation with anti-CD3/CD28 antibodies. Intracellular cytokine staining was performed by using monensin and brefeldin (BioLegend) and the cells were assessed for the production of IL-2, TNFα and IFNγ. Similarly, the BMDMs were separately analyzed for the expression of costimulatory ligands like 4-1BBL and CD38 of M1 macrophages. All flow cytometry data were acquired on a Fortessa X 20 (BD Biosciences, CA) and analyzed by using FlowJo (Treestar, OR).

CAR-T cell preparation. The MSGV1 γ retroviral vector backbone was modified to express CEA specific scFv, as described in our previous study. Retroviral supernatants produced from CEA expressing modified MSGV1 transduced PLAT-E cell line was collected. Briefly, CD8⁺ T cells isolated from B6 Thy 1.2 mice were transduced with the viral supernatants containing CEA in the presence of 5 μg/ml Polybrene (Sigma Aldrich, USA), following a protocol as described previously. The transduced cells were positively identified by expression of c-myc.

Animal Experimentation. The 6-8 weeks old wild-type (WT) C57BL/6 (B6) Thy 1.1 mice (Jackson Laboratories) were subcutaneously injected with 1×10⁶ MC32 CEA cancer cells in the right lateral flank on Day 0. Subsequently, the mice were divided into 3 different groups (n=5) with each group receiving either 1×PBS (Ctrl), heat killed bacteria (HK) or live attenuated bacteria (Live) on Day 9 post inoculation of tumor cells. On Day 12 post induction of tumor, all the groups of mice received the CEA CAR-T cells isolated and prepared from 6-8 weeks old WT C57BL/6 (B6) Thy 1.2 mice (6-8 weeks old, male; Jackson Laboratories) on Day 12 post induction of tumor.

Fluorescence imaging of BmΔvjbR in macrophages. BMDMs were seeded on glass coverslips in 24-well plates and inoculated with BmΔvjbR. At 1, 4, and 24 hours post-inoculation, the cells were washed 3 times with 1×PBS and fixed with 4% paraformaldehyde (in 1×PBS) for 15 minutes at room temperature. The fixed cells were then washed with 1×PBS 3 times and permeabilized with 1% Triton X-100 for 15 minutes and blocked with 5% bovine serum albumin in 1×PBS for 30 minutes. Subsequently, cells were stained with a rabbit anti-Brucella antibody (1:500 dilution, Bioss Inc.) for 1 hour and the secondary donkey anti-rabbit IgG-CF568 antibody (1:1,000 dilution, Biotium, Inc.) for 1 h. Cells were then mounted with ProLong Glass Antifade Mountant with NucBlue™ Stain (Thermo Fisher Scientific). For staining bacteria in tumor tissue, formalin fixed, paraffin-embedded sections of MC32 tumor tissue were sectioned at 4 μm and placed on charged glass slides. The sections were deparaffinized in xylene and rehydrated through graded alcohols. Antigen retrieval was performed in a pressure cooker (Decloaking Chamber, Biocare Medical, Pacheco, CA) using a citrate buffer. The tissues were stained by adopting a similar procedure as cells staining. All the images were acquired using a Nikon Eclipse Ti2 fluorescence microscope.

Bacterial quantification. For detecting BmΔvjbR survival in macrophages, BMDMs, J774A.1, or RAW 264.7 were seeded in the wells of 24-well plates at a concentration of 2.0×10⁵ cells/well in 1.0 mL of DMEM without antibiotics. After overnight culture, the cells were inoculated with live BmΔvjbR bacteria at a MOI of 20. The bacteria inoculation was followed the procedure described above. At 1, 4, and 24 hours post inoculation, cells were washed 3 times with 1×PBS and lysed in 0.5% of Triton X-100 for 30 minutes, and serial dilutions of the cell lysate was subjected to a serial of 10× dilution into 1×PBS and 10 μl of diluted cell lysates were streaked across TSA plates. The CFU of inoculated bacteria was also assayed by a serial dilution spotting on TSA plates. The plates were incubated at 37° C. for 3 days before the enumeration of CFUs. For CFU assay of BmΔvjbR in different tissues of cancer bearing mice, the mice were sacrificed at 19 days post injection, and lung, spleen, kidney, liver, and tumor tissues were collected and homogenized separately. The homogenates were serially diluted and spotted on TSA plates for CFU counting as above.

Comparative metabolic analysis. The differences in the glycolytic states of CD8⁺ T cells were analyzed using extracellular flux (XF) analyzers (Agilient) using a protocol described previously. Briefly T cells in suspension were removed from the co-cultured medium and seeded on 96-well seahorse plates. Their extracellular flux and compensatory glycolysis were assessed by using glycolytic activators and inhibitors.

Imaging and immunohistochemistry of tumor sections. Paraffin embedded solid tumor samples were sliced into 5 μm sections with microtome. The slides that were prepared from these sections were processed for fluorescence microscopy, Hematoxylin and Eosin (H&E) staining and mass cytometry analysis.

Imaging mass cytometry analysis. Mass Cytometry analysis of tumor samples derived from BmΔvjbR treated mice or controls were processed for the quantification, imaging, and analysis of DNA, Ki67 antigen, CD8⁺ T cells, B220 (B cells), CD11b (dendritic cells) and F4/80 (macrophages) respectively. A dimensionality reduction technique was adopted to construct t-Distributed Stochastic Neighbor Embedding (t-SNE) plots from the heatmaps of treated or untreated groups of mice. The neighborhood analysis was constructed to find the probability of enriched cell to cell interactions using basic statistical methods.

RNA isolation, cDNA preparation, and qPCR analysis. At 24 hours post treatment of BMDMs, the cells were washed twice with cold DPBS, and lysed in Trizol reagent. RNA was extracted using Direct-zol RNA Miniprep Kits (Zymo Research) following the manufacturer's protocol. For cDNA preparation, cDNA was synthesized from isolated RNA (1 μg/reaction) using High-Capacity cDNA Reverse Transcription Kit (Thermo Fisher Scientific) according to manufacturer's protocol. The quantification of mouse cytokines was performed using TaqMan Array 96-Well Fast Plates with TaqMan Fast Advanced Master Mix. The PCR reaction was run in StepOnePlus Real-Time PCR System (Applied Biosystems). Gene expression was analyzed by using ΔΔCT Method.

### Example 2

### BmΔvjbR Induces Anticancer Phenotypes in Bone Marrow-Derived Macrophages (BMDMs)

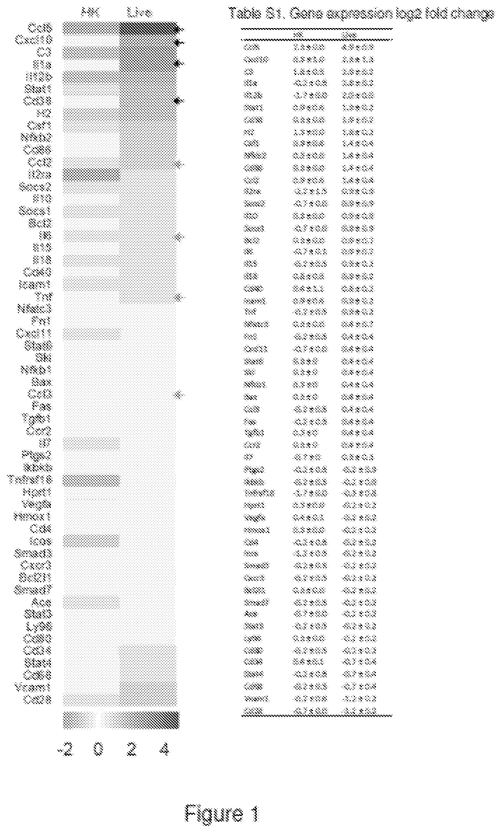

The instant example provides several experiments to characterize the anticancer potential of BmΔvjbR. First, to test the hypothesis that BmΔvjbR elicits anti-cancer pro-inflammatory phenotypes from immune cells, the live attenuated strain with murine BMDMs was incubated for 24 hours, and then used TaqMan qRT-PCR arrays to interrogate the gene expression of immune-related genes. It was found that live BmΔvjbR, but not heat-killed (HK) controls, induced BMDMs to express pro-inflammatory cytokines and chemokines including IL6, IL1α, IL12b (IL12p40), Cc15 (RANTES), Cxc110 (IP-10), Ccl2 (MCP-1), and Cc13 (MIP-1α) (FIG. 1).

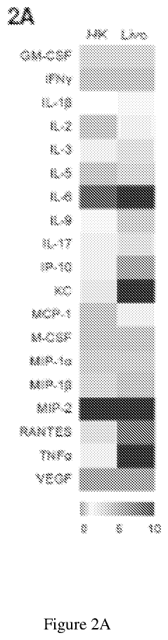

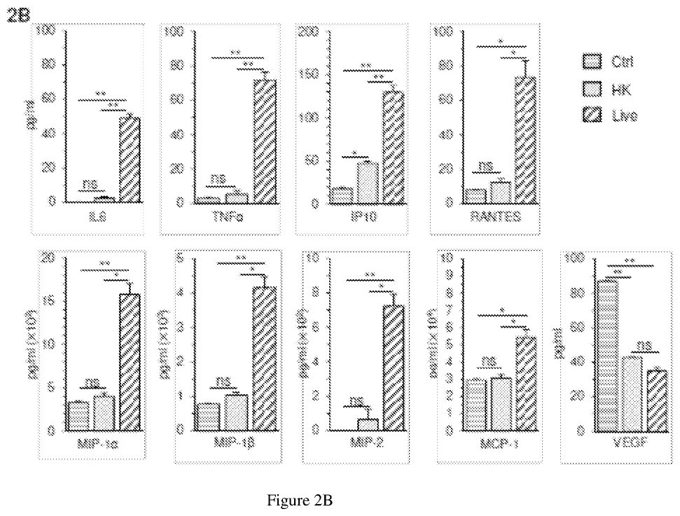

Second, to test the hypothesis that cytokine expression profiles followed the same trends as the gene expression profiles in this system, cytokine arrays and quantitative ELISA technology were used to measure cytokine production in BMDM culture medium at 24 hours post-treatment with live BmΔvjbR. It was found that the cytokine production profile corroborated the gene expression profile, with induced secretion of proinflammation cytokines and chemokines in BMDMs treated with live BmΔvjbR (FIGS. 2A-2B) in contrast to HK or no treatment (Ctrl). Specifically, significant increases in T-cell chemo-attractants Cxc110 (IP-10), Cc15 (RANTES), and MCP-1 were observed, as well as cytokines and chemokines known to be produced by activated macrophages, including IL6 and TNFα. Interestingly, significant decreases in the level of vascular endothelial growth factor (VEGF) were observed after BMDMs were incubated with live BmΔvjbR. This protein is a key mediator of angiogenesis in cancer and is frequently associated with tumor development and metastasis. Collectively, without being bound by any theory, these data could suggest that BmΔvjbR could trigger the production of proinflammatory cytokines and T cell-mediated chemo-attractants, which are T cell activation and attraction factors, respectively.

Third, to test the hypothesis that BmΔvjbR polarizes immune cells toward proinflammatory states, flow cytometry was used to monitor BMDM polarization and costimulatory molecule expression. It was found that most BMDMs were polarized to M1 macrophages, which express CD38, an M1 exclusive marker, on their surface after incubation with live BmΔvjbR (FIGS. 2C-2D). In accordance with the protein expression data, CD38 gene transcription levels were also dramatically increased after incubation with the live bacterial strain (but not HK controls) (FIG. 1).

### Example 3

### BmΔvjbR Induces Anticancer Phenotypes in CD8⁺ T Cells

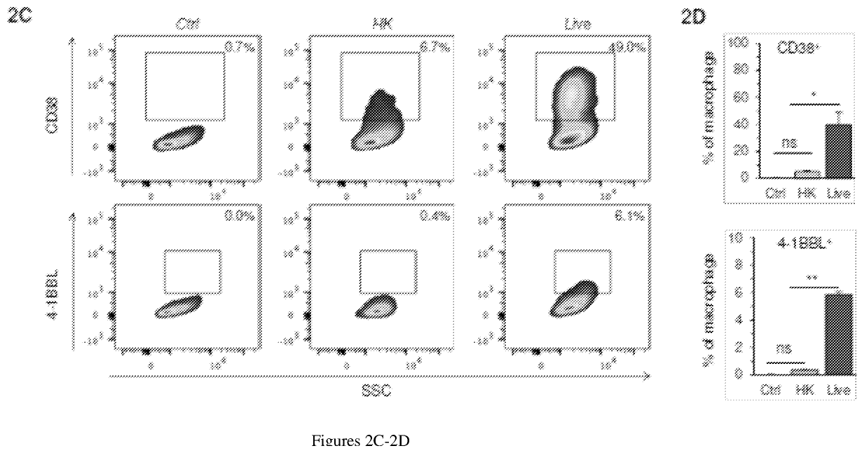

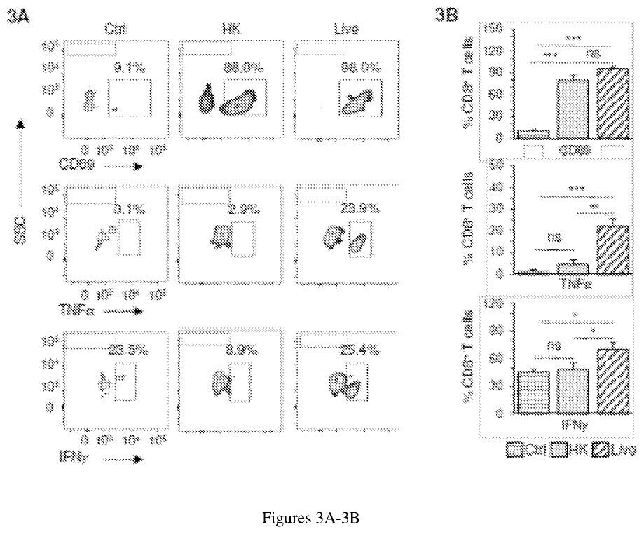

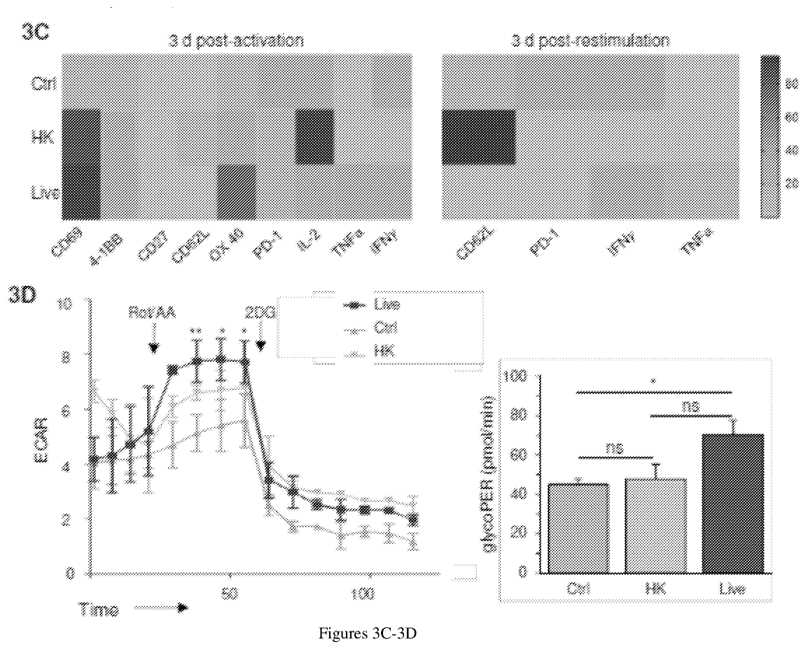

The instant example provides examples to examine whether BmΔvjbR can enhance the anti-cancer inflammatory potential of CD8⁺ T cells. To test the hypothesis that the live attenuated BmΔvjbR strain activated CD8⁺ T cells through polarization of macrophages (FIGS. 2C-2D), co-cultured CD8⁺ T cells with BMDMs pre-treated with either the live or HK bacteria were utilized. It was found that BMDMs exposed to BmΔvjbR activated the CD8⁺ T cells more efficiently compared to controls through upregulation of activation marker CD69, and induced significantly higher production of TNFα, IFNγ and IL-2 from CD8⁺ T cells (FIGS. 3A-3C). Moreover, the costimulatory marker expression, including OX40 and 4-1BB, were higher in CD8⁺ T cells co-cultured with BmΔvjbR treated BMDMs (FIG. 3C). The CD8⁺ T cells recall responses are critical for their antitumor efficacy. To test the hypothesis that the activated CD8⁺ T cells retained their functional recall ability, anti-CD3/anti-CD28 antibodies were used to restimulate CD8⁺ T cells 3 days post-activation. It was found that the CD8⁺ T cell recall responses were enhanced 3 days post-restimulation exhibiting lower PD-1 expression and higher expression of pro-inflammatory cytokines, including TNFα and IFNγ (FIG. 3C). CD8⁺ T cells also had a significantly higher extracellular acidification rate (ECAR) and showed higher glycolysis activity when activated with BMDMs treated with live or HK BmΔvjbR, indicating the highly activated CD8⁺ T cell phenotype (FIG. 3D). Taken together without being bound by any theory, the results could suggest that the activity and metabolism of CD8⁺ T cells is greatly enhanced in the presence of Bm. ΔvjbR treated macrophages.

### Example 4

### BmΔvjbR Induces Diverse Cellular Responses

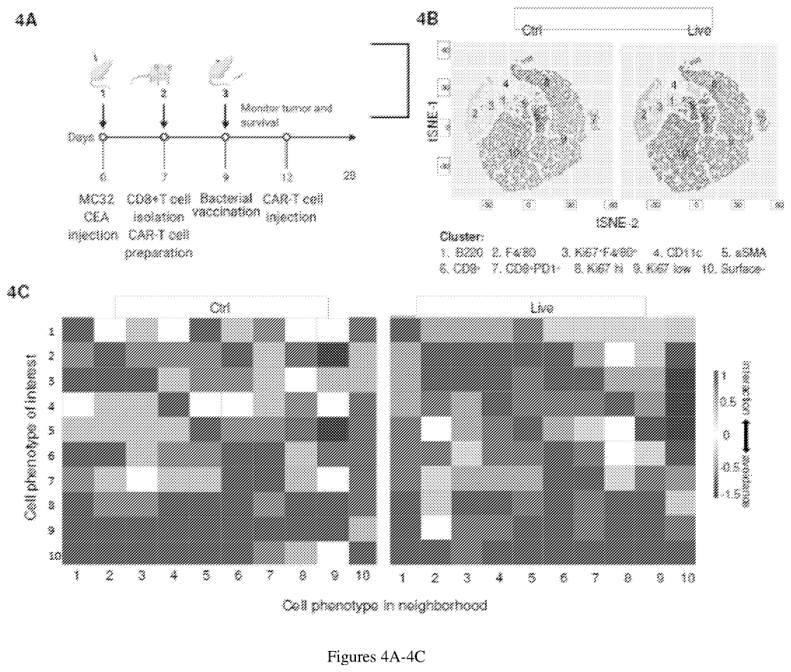

Having pursued experiments that show BmΔvjbR could be utilized to enhance the function and anti-cancer potential of BMDMs and CD8⁺ T cells, the instant example provides experiments to demonstrate if BmΔvjbR treatment could be utilized to alter the tumor microenvironment (TME) in an in-vivo murine solid-tumor model system were performed. Imaging mass cytometry (IMC) analysis to quantify the abundance of B cells and proliferating as well as non-proliferating immune cells from explanted solid tumor sections was performed. The experimental scheme for these studies involved sub-cutaneous inoculation of MC32 CEA colon cancer cells in the right lateral flank of Thy 1.1 C57BL/6 mice (FIG. 4A). On day 7 post-induction of tumor, CD8⁺ T cells were isolated from Thy 1.2 C57BL/6 mice and transduced to generate carcinoembryonic Ag (CEA) CAR-T cells. The experimental group of mice (n=5) were treated with either live or HK BmΔvjbR, whereas the control group were sham treated with PBS (n=5) (FIG. 4A). It was found that live BmΔvjbR treated mice had higher complexity of immune cells in the TME (FIGS. 4B-4C) compared to controls. To determine the significantly enriched interactions between or within the cell phenotypes in the TME, neighborhood joining plots from the CyTOF data were constructed. The tSNE plots (FIG. 4B) and neighborhood joining analysis (FIG. 4C) showed that the innate immune cells were activated and quantitatively higher in the TME of mice receiving the treatment. The reconstructed image from the mass-cytometry analysis showed more immune cells, especially F4/80⁺ macrophages, in the TME of BmΔvjbR treated mice receiving adoptive transfer of CAR-T cells (FIG. 4D). Therefore, the specific innate immune cells were quantified from the TME, and it was found that the numbers of Ki67-F4/80⁺ (non-proliferating macrophages), Ki67⁺F4/80⁺ (proliferating macrophages), and CD11c⁺ (dendritic cells) were significantly increased in BmΔvjbR treated mice receiving adoptive transfer of CAR-T cells (FIG. 4E). Moreover, the CD8⁺ PD-1-T cells were also high in the TME of the mice receiving BmΔvjbR compared to control. Overall, without being bound by any theory, these results could indicate that numbers of macrophages and dendritic cells were significantly increased in the TME of treated mice receiving adoptive transfer of CAR-T cells, consistent with the hypothesis that these immune cells could promote CAR-T tumor infiltration and drive tumor regression in these animals.

### Example 5

BmΔvjbR treatment enhances antitumor efficacy

The instant example provides experiments to demonstrate if BmΔvjbR treatment could enhance the antitumor efficacy of CAR-T cell therapy were performed. It was found that BmΔvjbR treated mice displayed significantly greater survival (FIG. 5A) and had drastically lower tumor burden than controls (FIG. 5B). Furthermore, hematoxylin and eosin (H&E) staining confirmed the significantly lower tumor burden in these mice (FIG. 5C). To investigate the tissue distribution of CD8⁺ T cells in BmΔvjbR treated animals, the abundance of Thy 1.2 CD8⁺ T cells inside the dissociated tumor using flow cytometry and confocal microscopy was measured. It was found that there were significantly increased numbers of CD8⁺ T cells infiltrating into the solid tumor of mice live BmΔvjbR treated in comparison to controls (FIGS. 5D-5E).

### Example 6

### BmΔvjbR Selectively Colonize Tumor Tissue

The instant example provides experiments to measure BmΔvjbR clearance from treated mice. After 19 days of post intravenous injection, tissues from tumor and other organs were homogenized for colony forming unit (CFU) assays. Tumor tissue was also fixed for immunofluorescence microscopy analysis. BmΔvjbR in tumor tissue was found (FIG. 5F) but not in other organs (FIG. 5G). The survival of BmΔvjbR in macrophages in vitro using immunofluorescence staining and CFU enumeration was also monitored. Using immunofluorescence microscopy, numerous bacterial cells in BMDMs were found at 1 hour and 4 hours post-inoculation. However, fewer were observed at 24 hours (FIG. 5H). Importantly, live bacteria were only recovered from BMDMs at 1 hour post infection, and no bacteria survived longer than 24 hours post inoculation in BMDMs, J774A.1, and RAW 264.7 (FIG. 5I). Without being bound by any theory, these results could indicate the BmΔvjbR strain selectively targeted the tumor, survived for only short times in macrophages, and were rapidly cleared from non-tumor tissue after treatment.

The instant example reports that BmΔvjbR activates CD8⁺ T cells and macrophages and disrupts the TME in favor of a reinvigorated immune environment characterized by increased cytokine production (TNFα and IFNγ). The TME harbors interactions between tumor cells and surrounding cells that contribute to the development and progression of cancer. Importantly, cancer cells express factors that suppress immune surveillance and cancer clearance in the TME, thereby creating a permissive environment for the uncontrolled proliferation of cancer cells. In this instant example, a novel and safe live attenuated bacterial strain BmΔvjbR could be utilized to remodel the TME to a pro-inflammatory status, and thereby limit cancer progression and tumorigenesis. Moreover, BmΔvjbR treatment, when combined with the adoptive transfer of antigen specific CD8⁺ T cells, could result in dramatically impaired tumor growth and proliferation. Therefore, this live attenuated bacterial strain could be utilized to potentiate immune surveillance and control of cancer.

Previous studies have demonstrated that treatment with live attenuated bacteria can limit tumorigenesis using a variety of mechanisms, and some of these bacterial approaches have entered clinical trials. For example, bacterial vectors such as Listeria, Salmonella and Lactobacillus have been investigated with varying levels of success in the promotion of overall antitumor immunity via direct cancer cell cytotoxicity, enhancement of cancer-specific immunity, and general immunomodulatory effects. In addition, a number of these bacterial vectors have been engineered to express augmented effector features that may promote anti-cancer immunity. All previously used bacterial vectors have intrinsic deleterious or toxic features, and suboptimal safety profiles or routes of delivery that may significantly limit their broad utility in cancer therapy/treatment. Among the negative features observed are intraperitoneal route of delivery persistence of viable microbes in non-cancerous tissues, significant endotoxin activity, pathogenic reversion potential, and limitations due to pre-existing host immunity. So far, there is no evidence to suggest that BmΔvjbR possesses the common deleterious properties shared by many of the previously studied bacterial vectors. Moreover, this work provides the first description of combining live attenuated bacterium treatment in the context of CAR-T therapy, and thereby demonstrates the synergy that can be achieved with these approaches.

Despite these advances, more selective and robust improvements in live attenuated bacterial treatment strategies could be envisioned based on our growing knowledge of the TME and the variety of mechanisms that can be targeted. Given the strong clinical efficacy of several novel cancer immunotherapies that prevent PD-1- or CTLA4-mediated checkpoint negative regulation of antitumor T-cells, bacterial anticancer vectors that achieve the same checkpoint inhibition without the need for biologic/antibody-mediated immunotherapy are highly desirable. Specifically, bacterial strains that induce lower levels of checkpoint proteins on the surfaces of immune cells may constitute attractive tools for addressing the immunosuppressive features of the TME. Toward this end, this instant application has shown that an important molecular mechanism by which BmΔvjbR limits tumorigenesis is by suppressing the expression of PD-1 on CD8⁺ T-cells.

### Example 7

### Exemplary Experimental Procedures

The instant example provides exemplary materials and methods utilized in Examples 8-10 as described herein.

Collagen-induced arthritis (CIA) model induction. CIA was induced when male C57BL/6 mice were injected with an emulsion of 100 μl of chick type II collagen (Chondrex; 100 μg) in Complete Freund's Adjuvant (CFA; Chondrex) using a glass tuberculin syringe with 26-gauge needle. The mice were then assessed for development of joint inflammation and clinical arthritis score until Day 60.

Bacterial culture. BmΔvjbR or BmΔvjbR::tnaA were cultivated and prepared for experimentation as previously described.

Engineering indole-producing BmΔvjbR::tnaA strain. To generate an indole producing attenuated BmΔvjbR strain, an Escherichia coli (E. coli) tnaA gene was cloned into a broad range bacteria expression plasmid (pBBRIMCS6Y) as described in Fernandez-Prada C M et al., Infect Immun., 2003; 71 (4): 2110-9, herein incorporated by reference in its entirety. The plasmid was then transferred to BmΔvjbR.

Indole detection and quantification. The indole production by BmΔvjbR::tnaA was detected by liquid chromatography-mass spectrometry (LC-MS). After 24 hour cultivation in Tryptic Soy Broth (TSB) medium at 37° C., the metabolites were extracted using ice-cold methanol for LC-MS assay. Liquid chromatography tandem mass spectrometry analysis was performed on a TSQ Altis triple quadrupole mass spectrometer (Thermo Scientific, Waltham, MA) coupled to a binary pump HPLC (Vanquish, Thermo Scientific). The indole concentration was measured using indole assay kit (Sigma-Aldrich).

BmΔvjbR::tnaA treatment and ACT of T_{regs}. CIA was induced in male C57BL/6 mice. On Day 7 after the CIA induction, mice were intravenously (i.v). injected with 5.0×10⁷ live BmΔvjbR::tnaA or PBS control. In the BmΔvjbR::tnaA+T_{reg} combinatorial treatment group, mice (n=5) were adoptively transferred with 2.5×10⁶ CD4⁺ CD25⁺ T_{regs} derived from donor lymph nodes (LNs) and spleen of naive C57BL/6 mice, one week after the BmΔvjbR::tnaA administration.

Enumeration of BmΔvjbR::tnaA recovered from CIA mice. BmΔvjbR::tnaA (5.0×10⁷) were i.v. injected into C57BL/6 mice and the bacterial distribution and survival were analyzed by colony forming unit (CFU) assay. The mice were sacrificed at 1, 3, 7, 14, and 21 dpi of bacteria. The spleen, liver, LNs and joints were homogenized and plated on Tryptic Soy Agar (TSA) plates supplemented with chloramphenicol antibiotic. The CFU was enumerated after 3-day post-cultivation of the bacteria.

Serum ELISA for detection of BmΔvjbR specific IgG antibody. The CIA induced C57BL/6 mice were sacrificed at 1, 3, 7, 14, and 21 dpi of BmΔvjbR and/or BmΔvjbR::tnaA bacteria. Blood samples were collected from the mice and serum was isolated by coagulation of the blood at room temperature followed by centrifugation at 2,000×g for 20 minutes. The serum sample was assayed for anti-BmΔvjbR IgG antibody by using mouse Brucella antibody IgG ELISA kit (AFG Scientific).

Cytokine responses. BMDMs were seeded in 24-well plates at a concentration of 2.0×10⁵ cells/well in DMEM without antibiotics. After overnight culture, the cells were inoculated with BmΔvjbR or BmΔvjbR::tnaA bacteria at a multiplicity of infection of 20. At 24 hours post-treatment, cellular supernatant was collected and analyzed for the presence of cytokines/chemokines by using a Proteome Profiler Mouse Cytokine Array Kit (R&D Systems, Inc.).

Flow cytometric analysis. Cell staining and flow cytometric analysis were performed using the described labeling reagents. Briefly, surface or intracellular staining was performed on the single-cell suspensions and analyzed using LSR Fortessa cell analyzer (BD). The joints were also processed and stained similarly with antibodies listed in Table S2, and data was acquired on CyTEK aurora flowcytometer (Cytek Biosciences). For multiparametric analyses, the data were analyzed with FlowJo v10 and represented as heatmaps and tSNE plots.

Histology and immunofluorescence. Mice were humanely sacrificed on day 60 after induction of CIA, and tissue sections were analyzed. Briefly, the hind foot paws and knees were removed and fixed in 10% formalin and decalcified in Formical-4 (Decal chemical, Tallman, NY). The fixed tissue sections were then stained with H&E and/or Safranin O fast green (Saf-O) stain. The H&E- and Saf-O-stained sections were then assessed by semiquantitative system of 0 to 4. Immunofluorescent staining and microscopy were performed on the deparaffinized sections by using FITC anti-mouse FoxP3 antibody (Ab) for T_{regs} and DAPI as nuclear stain.

### Example 8

### Indole and Immune-Mediated Inflammation

An attenuated strain of Brucella melitensis was selected for the bacterial vector that harbors a deletion in vjbR, a master regulator of virulence (BmΔvjbR). Like other Gram-negative organisms, Brucella strains express a lipopolysaccharide (LPS) lacking endotoxin activity. Importantly. BmΔvjbR is known to be safe in immunocompetent and immunocompromised mice, goats, sheep, and non-human primates. In the instant example, BmΔvjbR engineered to express tryptophanase (tnaA); i.e., BmΔvjbR::tnaA, produces the tryptophan metabolite indole, a molecule that modulates the fate and function of T_{regs}.

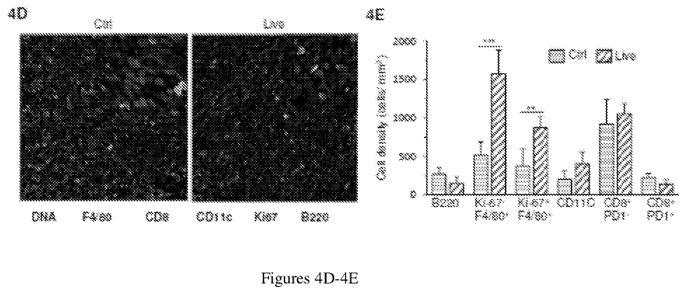

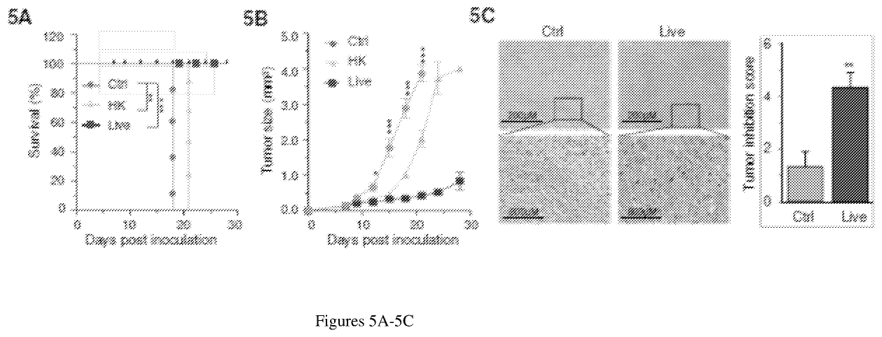

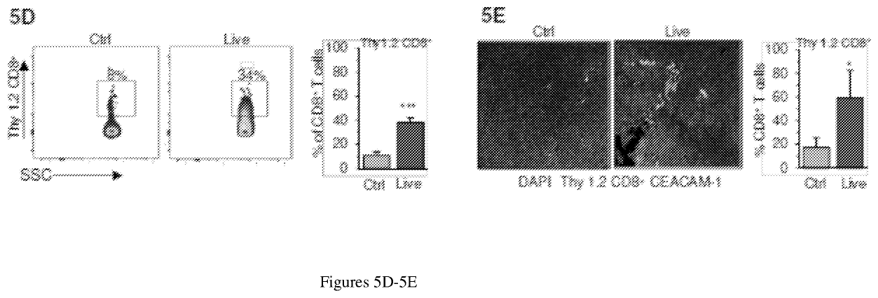

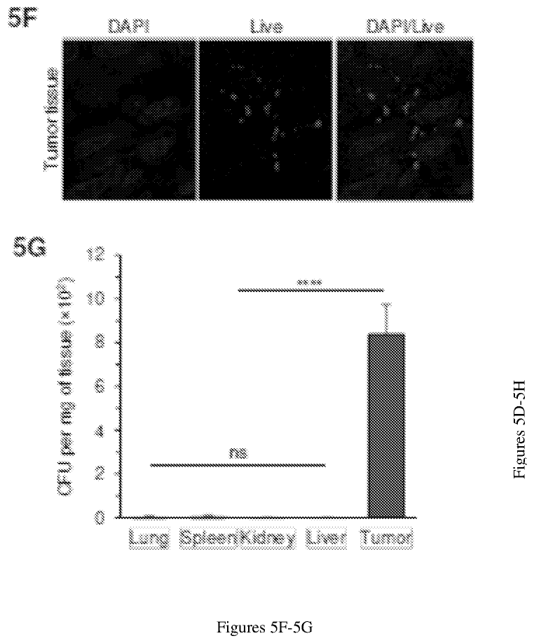

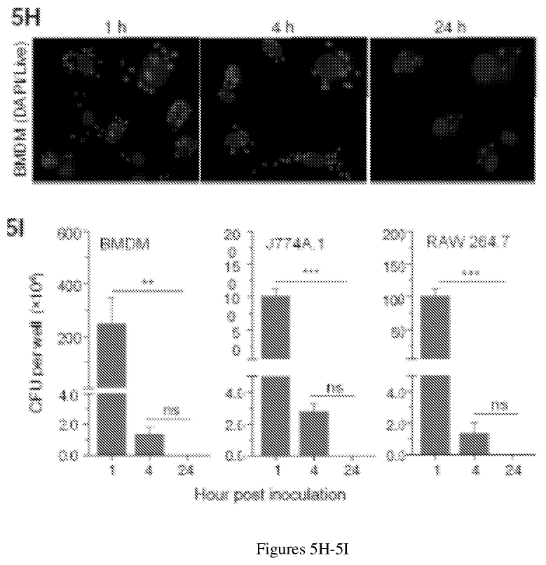

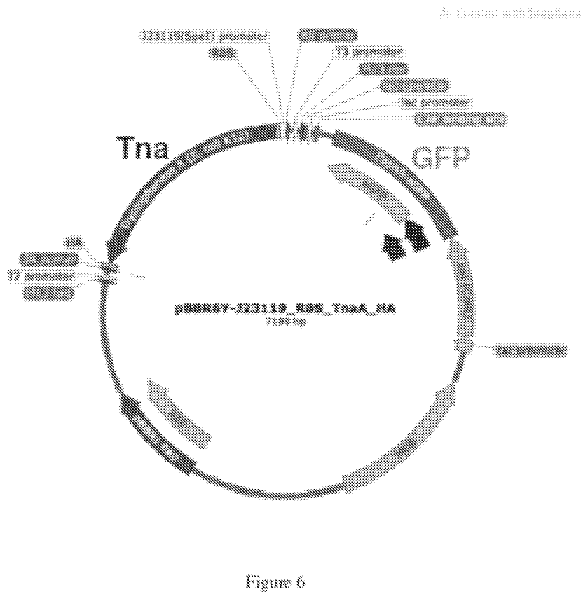

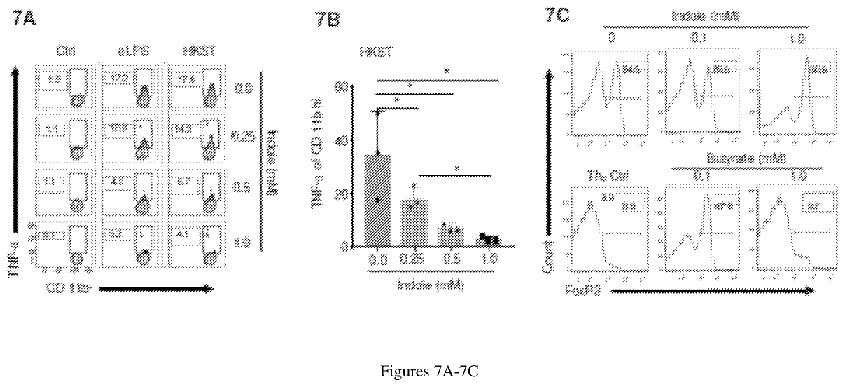

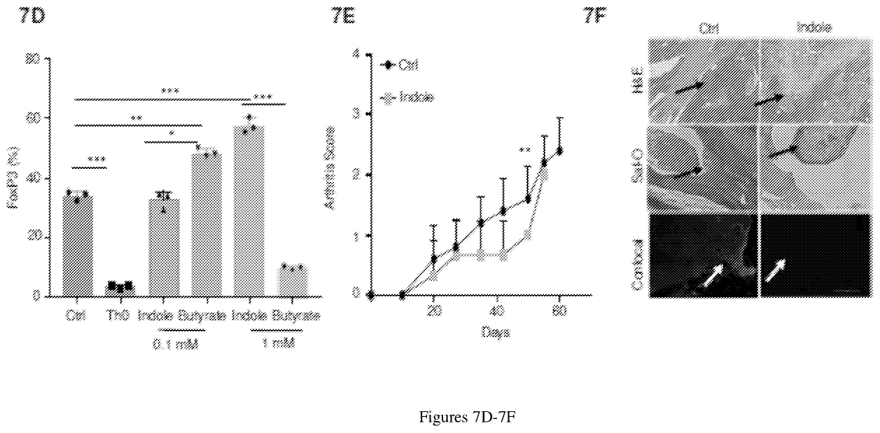

Indole is capable of suppressing several inflammatory characteristics in immune and non-immune cells and augments T_{reg} differentiation. As shown in FIGS. 7A and 7B, indole suppressed TNF-α production in CD11b⁺ spleen cells after E coli LPS (eLPS) and heat-inactivated Salmonella Typhimurium [HKST] stimulation. Further, indole and dampened their activation by suppressing Akt and ERK signaling pathways in response to microbial agonists (eLPS and HKST). In addition, indole augmented the differentiation of naive CD4⁺ CD25-T cells into induced T_{regs} (iTr_{egs}) measured by FoxP3 in vitro in a dose dependent manner (FIG. 7C & FIG. 7D). Without being bound by any theory, indole is believed to ameliorate immune-mediated inflammation in autoimmune and pro-inflammatory diseases.

### Example 9

### Indole Reduces Autoimmune Responses in Collagen-Induced Arthritis (CIA) Model

The instant example demonstrates that indole reduces autoimmune responses in a murine collagen-induced arthritis (CIA) model. First, the severity of CIA was significantly attenuated in indole treated mice, which exhibited clinical scores of 0.8±0.2 (means±SEM) at (Day 50), compared to 1.6±0.5 in controls (FIG. 7E). However, a single dose of indole only showed a slight decrease in inflammation.

Similarly, single dose treatment did not induce significant alterations in the infiltration of T_{regs} as assessed by confocal microscopy (FIG. 7F & FIG. 7G). These findings contrast with ex vivo experimental findings indicating that indole significantly promoted the expansion of CD4⁺ FoxP3⁺ T_{regs} and enhanced their activation by increased expression of PD-1 an immunosuppressive molecule, compared to the controls (p<0.001), in cells derived from the mouse lymph nodes (LNs) and spleen (FIG. 7H & FIG. 7I).

### Example 10

Delivery of Indole Via BmΔvjbR::tnaA

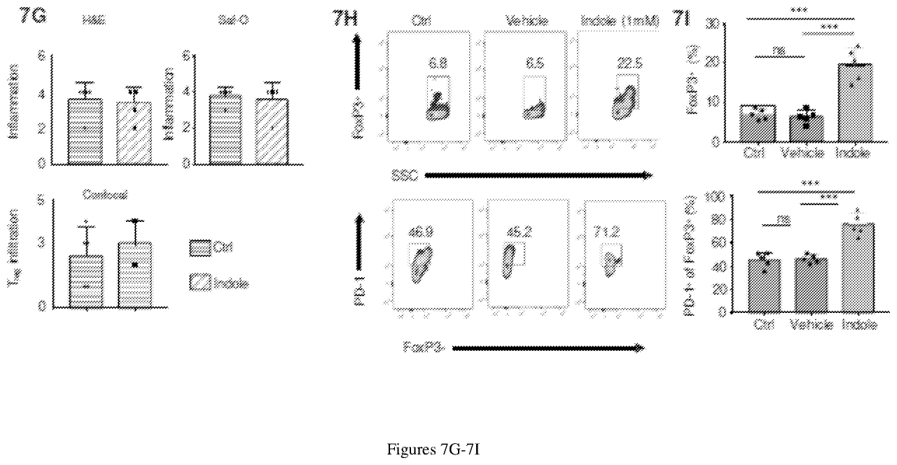

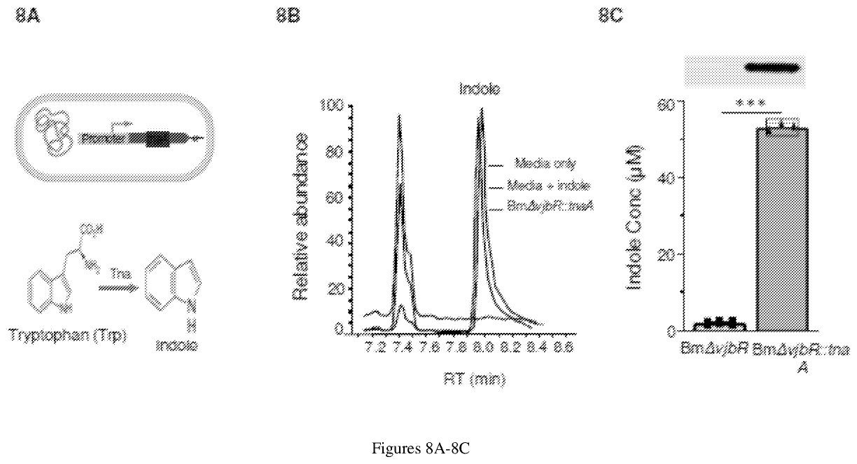

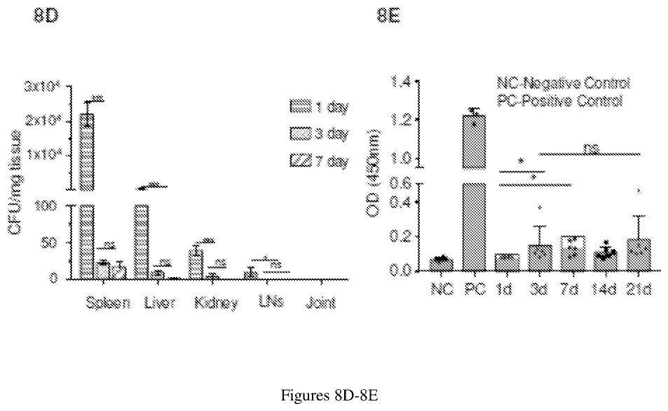

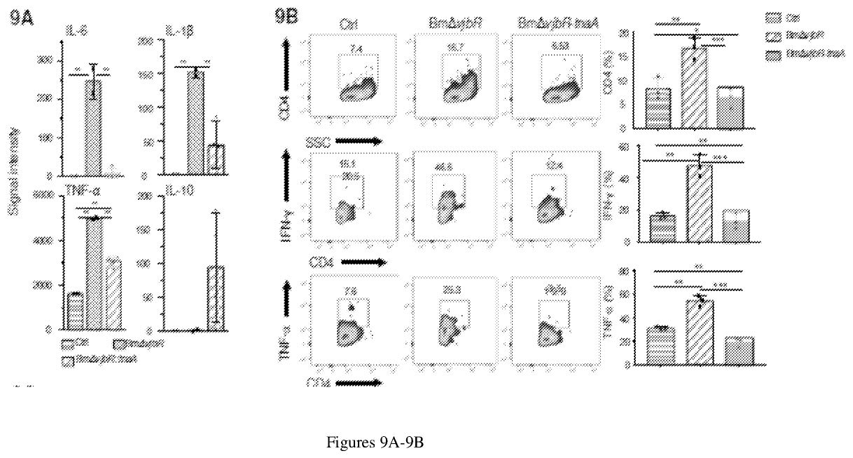

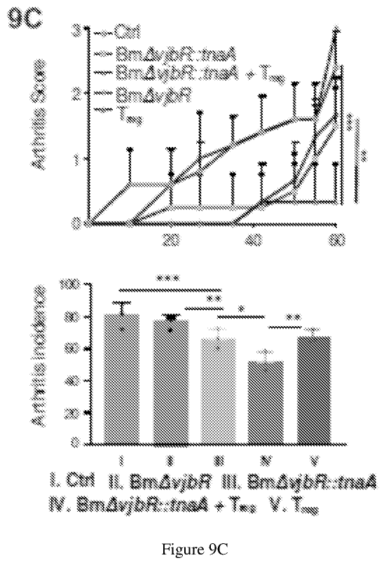

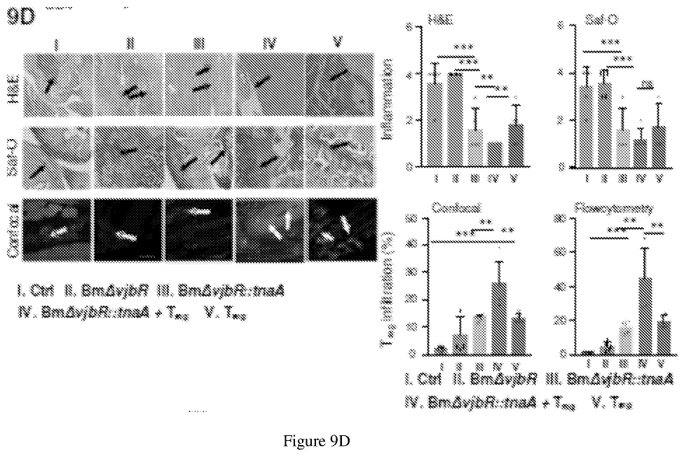

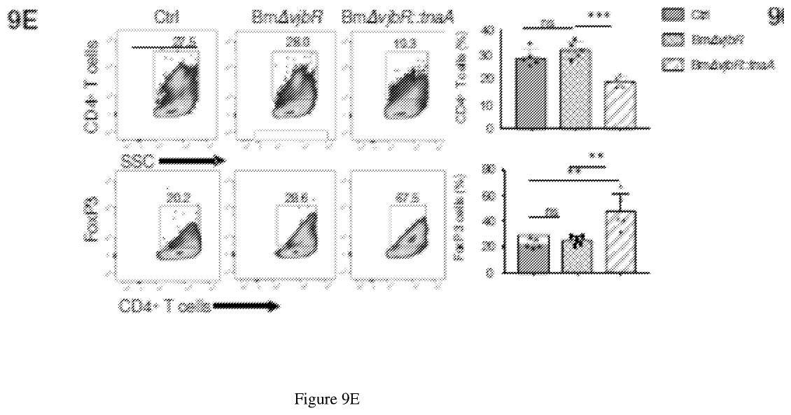

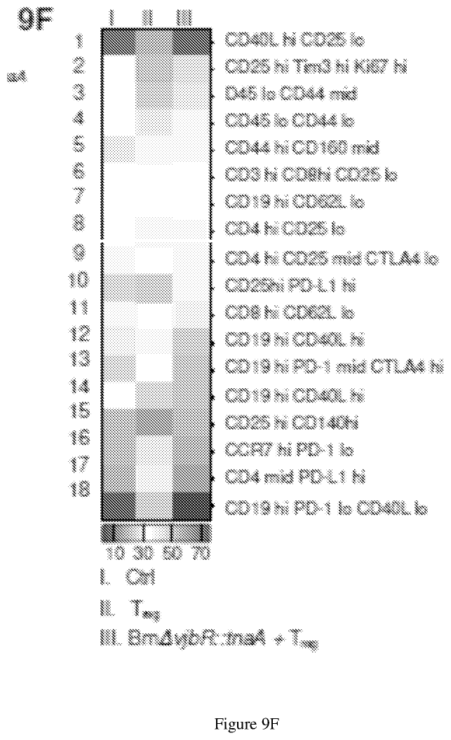

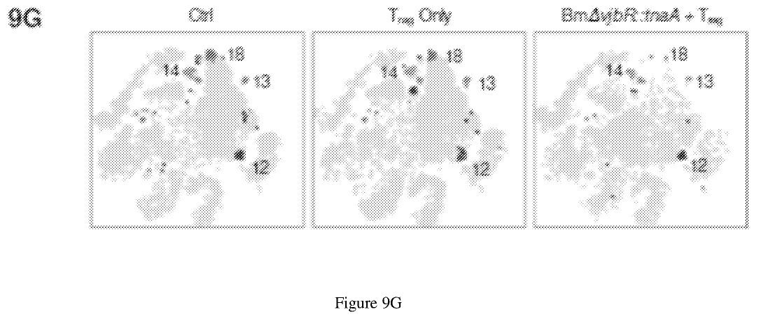

The instant example evaluated if the sustained delivery of indole in a bacterial vector affected the durability of the molecule's immunomodulatory effects, resulting in an attenuation of autoimmunity and inflammation in CIA. A safe, live-attenuated bacterial strain (i.e., BmΔvjbR::tnaA) was engineered to constitutively produce indole (FIG. 8A-8C). First, the engineered bacterial strain was observed to survive mainly in the spleen for 7 days and liver and kidney for 3 days post-inoculation (dpi) (FIG. 8D). The bacteria did not penetrate the joints of the mice (FIG. 8D). Second, low-level immunogenicity was observed via detection of anti-Brucella IgG antibody from 3 to 21 dpi of the bacteria (FIG. 8E). Moreover, it was determined that the BmΔvjbR::tnaA bacterial strain recovered from mice challenged with collagen-induced arthritis (CIA) at 7 dpi could still produce indole (50 to 70 μM). Further, cytokine array profiling analyses showed that BmΔvjbR::tnaA induced the expression of IL-10 (FIG. 9A), which promotes the activities of T_{regs} and reduces autoimmunity and inflammation.

Surprisingly, BmΔvjbR::tnaA also significantly (p<0.01) reduced the expression of additional pro-inflammatory cytokines such as IL-6, IL-1β, and TNF-α in macrophages (Mφ) compared to BmΔvjbR parental strain (FIG. 9A). In addition, BmΔvjbR::tnaA, when co-cultured with bone marrow-derived Mφ (BMDMs), not only significantly reduced the total CD4⁺ T cells (p<0.001) but also reduced the production of the pro-inflammatory cytokines such as TNF-α and IFN-γ (p<0.001) compared to the BmΔvjbR parental strain (FIG. 9B). BmΔvjbR::tnaA also promoted the expansion of T_{regs} and significantly enhanced their activity as assessed by IL-10 production (p<0.001) and PD-1 expression (p<0.01). Third, in the CIA model, a significant reduction in arthritis score and incidence was observed following treatment with BmΔvjbR::tnaA. This amelioration of autoimmunity and inflammation was further augmented when BmΔvjbR::tnaA treatment was combined with ACT of T_{regs} (FIG. 9C).

Significantly reduced numbers of infiltrating inflammatory cells (p<0.01) were observed into the joints of mice treated with BmΔvjbR::tnaA. This effect was further enhanced by BmΔvjbR::tnaA treatment followed by the ACT of T_{regs} compared to the controls (p<0.001) (FIG. 9D). Finally, mice treated with BmΔvjbR::tnaA showed reduced infiltrates in the joint as evidenced by H&E analysis and Safranin O (Saf-O) staining of knee cross-sections (60 days post collagen administration). Surprisingly, these findings were further attenuated by addition of ACT of T_{regs} (FIG. 9D). There was also a significant reduction in the total CD4⁺ T cell proportions and a dramatic increase in T_{reg} (p<0.001) proportions in mice treated with BmΔvjbR::tnaA compared to controls (FIG. 9E).

To identify the BmΔvjbR::tnaA mechanism of action, a multiparametric CyTEK analysis was conducted from the cells isolated from the joints of control, ACT with T_{reg} only, or ACT with T_{reg} plus BmΔvjbR::tnaA groups. BmΔvjbR::tnaA was observed to reduce the proportion of B cells (FIGS. 9F and 9G) in addition to promoting T_{reg} expansion. Overall, BmΔvjbR::tnaA can remodel the pro-inflammatory microenvironment and facilitates the expansion and suppressive function of T_{regs} and can also modulate B cell-mediated immunity in the CIA model.

1. A pharmaceutical composition comprising a live attenuated bacterial strain of Brucella melitensis.

2. (canceled)

3. (canceled)

4. (canceled)

5. (canceled)

6. The pharmaceutical composition of claim 1, wherein the pharmaceutical composition further comprises a second therapeutic agent.

7. (canceled)

8. (canceled)

9. (canceled)

10. The pharmaceutical composition of claim 1, wherein the pharmaceutical composition further comprises indole.

11. The pharmaceutical composition of claim 10, wherein the live attenuated bacterial strain of Brucella melitensis is modified to produce indole.

12. A method of treating a patient, said method comprising the step of administering a pharmaceutical composition comprising a live attenuated bacterial strain of Brucella melitensis to the patient.

13. (canceled)

14. The method of claim 12, wherein the patient is in need of treatment for cancer.

15. The method of claim 12, wherein the patient is in need of treatment for an autoimmune disorder.

16. The method of claim 12, wherein the patient is in need of treatment for an inflammatory disorder.

17. The method of claim 12, wherein the method elicits a CD8+ T cell response in the patient.

18. The method of claim 12, wherein the method elicits a CD4+ T cell response in the patient.

19. The method of claim 12, wherein the method elicits a T regulatory cell response in the patient.

20. The method of claim 12, wherein the method increases PD-1 expression on CD8+ T cells in the patient.

21. The method of claim 12, wherein the method increases the number of CAR-T cells in a tumor microenvironment of the patient.

22. The method of claim 12, wherein the method increases the activity of CAR-T cells in a tumor microenvironment of the patient.

23. (canceled)

24. (canceled)

25. (canceled)

26. (canceled)

27. (canceled)

28. The method of claim 12, wherein the method induces macrophages in the patient to express a pro-inflammatory cytokine/chemokine.

29. (canceled)

30. The method of claim 12, wherein the method induces reduction of VEGF in the patient.

31. The method of claim 12, wherein the method enhances inflammatory potential of CD-8+ T cells.

32. (canceled)

33. (canceled)

34. (canceled)

35. (canceled)

36. (canceled)

37. A transgenic attenuated Brucella melitensis strain, said strain comprising a mutation in a virulence gene of said strain, said mutation selected from the group consisting of vjbR, asp14, and mucR wherein said mutation inactivates the virulence gene; and a nucleic acid encoding tryptophanase (tnaA).

38. The transgenic attenuated Brucella melitensis strain of claim 37, wherein the mutated virulence gene is vjbR, and the nucleic acid encoding tnaA is expressed under the control of a constitutive promoter.

39. The transgenic attenuated Brucella melitensis strain of claim 37, wherein the mutated virulence gene is BmΔvjbR and the nucleic acid encoding tnaA comprises E. coli tnaA.
